(19) United States
(12) Patent Application Publication
(10) Pub. No.: US 2015/0147355 A1
(43) Pub. Date: May 28, 2015

(54) Compositions and Methods of Vaccination

(71) Applicant: YALE UNIVERSITY, New Haven, CT (US)

(72) Inventors: Akiko Iwasaki, Guilford, CT (US); Norifuma Iijima, New Haven, CT (US); Haina Shin, Hamden, CT (US)

(21) Appl. No.: 14/401,795

(22) PCT Filed: Mar. 8, 2013
(86) PCT No.: PCT/US2013/029965

§ 371 Date: Nov. 17, 2014

Related U.S. Application Data
(60) Provisional application No. 61/648,316, filed on May 17, 2012.

(51) Int. Cl. A61K 39/245; A61K 45/06; A61K 39/21

(52) U.S. Cl. CPC A61K 39/245; A61K 39/21; A61K 45/06; C12N 2710/16634
USPC 424/208.1; 424/231.1; 424/277.1; 424/274.1; 424/234.1; 424/184.1

(57) ABSTRACT

The present invention relates to a vaccination strategy that establishes protective immunity, including a protective memory T-cell population.

### CROSS-REFERENCE TO RELATED APPLICATION

This application claims priority to U.S. Provisional Application Ser. No. 61/648,316, filed May 17, 2012, the contents of which are incorporated by reference herein in their entirety.

### BACKGROUND OF THE INVENTION

Viral sexually transmitted infections (STIs) such as human immunodeficiency virus 1 (HIV-1) and herpes simplex virus 2 (HSV-2) account for considerable morbidity and mortality around the world. Strong preclinical evidence for the role of T cells in controlling viral STIs has led to the design of prophylactic vaccines that elicit systemic cellular immunity, and yet these vaccines have not been efficacious (McElrath & Haynes, 2010, Immunity 33:542-554; Koelle & Corey, 2008, Annu. Rev. Med. 59:381-395). Although systemic memory T cells can migrate freely through organs such as the spleen and liver, other parts of the body such as the intestines, lung airways, central nervous system, skin and vagina are restrictive for memory T cell entry (Woodland & Kohlmeier, 2009, Nature Rev. Immunol. 9:153-161). In the latter tissues, inflammation or infection is often required to permit entry of circulating activated T cells to establish a tissue-resident memory T cell pool that is separate from the circulating pool (Gebhardt et al., 2009, Nature Immunol. 10:524-530; Masopust et al., 2010, J. Exp. Med. 207:553-564; Klonowski et al., 2004, Immunity 20:551-562).

Most successful existing vaccines rely on neutralizing antibodies, which may not require specific anatomical localization of B cells. However, efficacious vaccines that rely on T cells for protection have been difficult to develop, as robust systemic memory T cell responses do not necessarily correlate with host protection (McElrath & Haynes, 2010, Immunity 33:542-554). In peripheral sites, tissue-resident memory T cells provide superior protection compared to circulating memory T cells (Gebhardt et al., 2009, Nature Immunol. 10:524-530; Jiang et al., 2012, Nature 483:227-231). Given that side effects of inflammation in the reproductive tissue may preclude the use of a live prophylactic vaccine given vaginally, an alternative regimen to recruit virus-specific T cells into the vaginal mucosa without inducing local inflammation or infection is desirable.

Thus, there is a need in the art for a non-inflammatory vaccination strategy against viral sexually transmitted infections that relies on T cells. The present invention addresses this unmet need in the art.

### SUMMARY OF THE INVENTION

The present invention relates to a vaccination strategy for establishing protective immunity within a tissue, including an immunologically restrictive tissue. In one embodiment, the invention is a method of inducing an immune response in a subject, and recruiting the immune response to an anatomic location of the subject, including the steps of parenterally administering to the subject at least one immunogen, wherein the at least one immunogen induces an immune response, and locally administering to the anatomic location of the subject at least one chemokine, wherein the chemokine recruits the immune response to the anatomic location.

In some embodiments, the parenteral administration of the at least one immunogen is at least one selected from the group consisting of subcutaneous administration, intravenous administration, intramuscular administration, and intradermal administration. In some embodiments, the local administration of the at least one chemokine is at least one selected from the group consisting of topical administration, subcutaneous administration, intramuscular administration, intradermal administration, intracranial administration and intratumoral administration. In various embodiments, the chemokine is at least one selected from the group consisting of CXCL9, CXCL10 and CCL5.

In some embodiments, the anatomic location is an immunologically restrictive tissue. In some embodiments, the anatomic location is at least one selected from the group consisting of the genital mucosa, a tumor, the skin, the central nervous system, the peripheral nervous system, the testes, the placenta, the eye, the intestine, and the lung airways.

In one embodiment, the immunogen is derived from a cancer cell. In another embodiment, the immunogen is a derived from a tumor. In other embodiments, the immunogen is derived from a pathogen selected from the group consisting of a virus, a bacterium, a fungi and a protozoan. In some embodiments, the immunogen is at least one component of at least one selected from the group consisting of a live pathogenic organism, a live attenuated pathogenic organism, an inactivated pathogenic organism, and a dead pathogenic organism. In one embodiment, the immunogen is at least one selected from the group consisting of a peptide, a polypeptide, and a polynucleotide encoding a polypeptide. In some embodiments, where the immunogen is a polynucleotide encoding a polypeptide, the polynucleotide is RNA or DNA. In some embodiments, where the immunogen is a polynucleotide encoding a polypeptide, the polynucleotide is a DNA vaccine.

In some embodiments, the subject is not currently infected with the pathogen and the immune response is a protective immune response. In other embodiments, the subject is currently infected with the pathogen and the immune response is a therapeutic immune response. In some embodiments, the subject does not currently have cancer and the immune response is a protective immune response. In other embodiments, the subject currently has cancer and the immune response is a protective immune response.

In one embodiment, the immune response comprises a humoral immune response. In some embodiments, the immune response comprises at least one antibody. In some embodiments, the immune response comprises at least one antibody that specifically binds to the immunogen.

In one embodiment, the immune response comprises a cell-mediated immune response. In some embodiments, the immune response comprises at least one activated immune cell. In some embodiments, the activated immune cell is a CD4+ T cell. In other embodiments, the activated immune cell is a CD8+ T cell.

In some embodiments, the activated immune cell is a CXCR3+ T cell. In one embodiment, the activated immune cell is a CXCR3+CD4+ T cell. In another embodiment, the activated immune cell is a CXCR3+CD8+ T cell.

In some embodiments, the activated immune cell is a CCR5+ T cell. In one embodiment, the activated immune cell is a CCR5+CD4+ T cell. In another embodiment, the activated immune cell is a CCR5+CD8+ T cell. In some embodiments, the subject is human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1D, depicts a schematic and results of example experiments evaluating how effector T cells are recruited to the vagina by topical chemokine treatment. FIG. 1A: Experimental schematic. Donor gBT-I CD8⁺ T cell recipients were not immunized (naive), or were immunized either intravaginally (ivag) or subcutaneously (s.c.) with TK⁻ HSV-2. Five days post infection, subcutaneously immunized mice were treated vaginally with either the chemokines CXCL9 and CXCL10 (pull) or PBS. FIG. 1B: The frequency of donor gBT-I CD8⁺ T cells 1 day post pull in the indicated tissues (ILN, iliac lymph nodes). Plots are gated on total CD8⁺ T cells and numbers indicate per cent gBT-I (CD45.1⁺). FIG. 1C: The number of donor gBT-I CD8⁺ T cells 1 day post pull in the indicated tissues. FIG. 1D: The number of CD4⁺ T cells 1 day post pull in the vagina. FIGS. 1C, 1D: Numbers in graphs indicate fold difference in T cell number for subcutaneously immunized compared with subcutaneously immunized plus pull. Statistical significance was determined by two-tailed unpaired Student's t-test. Data are pooled from 2-7 independent experiments (n=6-21 per group). All error bars show s.e.m. and *P=0.05-0.01, **P=0.01-0.001, ***P<0.001 throughout all figures. FIG. 1C: *p=0.0225 (ivag vs. s.c.+pull) and **p=0.0019 (s.c. vs. s.c.+pull). D, **p=0.0037 (ivag vs. s.c.+pull), **p=0.0082 (s.c. vs. s.c.+pull)

FIG. 2, comprising FIGS. 2A through 2D, depicts the results of experiments demonstrating that the chemokine pull is specific for highly-activated effector T cells. Mice were subcutaneously immunized and given chemokines (CXCL9 and CXCL10) or PBS at day 5, 15 or 28 post infection and analyzed 1 day post pull. FIG. 2A: CXCR3 expression on donor gBT-I CD8⁺ T cells or CD44⁺CD4⁺ T cells from the spleen 1 day post pull from subcutaneously immunized mice (darker open line) and subcutaneously immunized plus pull (lighter open line). Shaded histograms are CD44^{1o} CD8⁺ or total CD4⁺ T cells. FIG. 2B: The gBT-I CD8⁺ T cell number in the vagina (left) or spleen (middle) and frequency in the spleen (right) were examined 1 day post pull. FIG. 2C: The number of CD44⁺CD4⁺ T cells in the vagina 1 day post pull on the indicated days post infection. FIG. 2D: The number of endogenous CD44⁺CD8⁺ T cells in the vagina 1 day post pull on the indicated days post infection. FIGS. 2B, 2C, and 2D: Statistical significance was determined by two-tailed unpaired Student's t-test. Data are pooled from 2-3 independent experiments; n=6-18 per group. FIG. 2B: In the vagina, *p=0.0171 (naïve vs. d5), *p=0.0399 (d5 vs. d15) and *p=0.0376 (d5 vs. d28). In the spleen, ***p<0.0001 (naïve vs. d5), **p=0.0016 (d5 vs. d15) and **p=0.0025 (d5 vs. d28). For frequency, **p=0.0023 (naïve vs. d5) and ***p<0.0001 (d5 vs. d15, d5 vs. d28). FIG. 2C: *p=0.0207 (naïve vs. d5), *p=0.0438 (d5 vs. d28). FIG. 2D: ***p<0.0001 (naïve vs. d5), **p=0.0033 (d5 vs. d15) and ***p=0.0009 (d5 vs. d28)

FIG. 3, comprising FIGS. 3A through 3E, depicts the results of experiments demonstrating that virus-specific T cells recruited by chemokine pull are retained in the vagina long term. FIG. 3A: Mice were immunized and treated as shown in FIG. 1A. At 4 weeks post pull, numbers of gBT-I CD8⁺ T cells were determined in the indicated tissues. The number inside the graph shows fold difference in gBT-I number between subcutaneously immunized compared to subcutaneously immunized plus pull groups. FIG. 3B: Four weeks post pull, the frequency of gBT-I cells was measured in the vagina. Plots are gated on total CD8⁺ T cells. Numbers show per cent gBT-I (CD45.1⁺). FIG. 3C: The number of endogenous CD4⁺ T cells in the vagina at 4 weeks post pull. FIG. 3D: The numbers of gBT-I cells were determined in the vagina at 12 weeks post pull (left) and compared to numbers at 4 weeks post pull (right). Number×ND is the number of animals with no cells detected in the tissue. FIG. 3E: The numbers of CD4⁺ T cells were determined in the vagina at 12 weeks post pull (left) and compared to the corresponding numbers at 4 weeks post pull (right). Statistical significance was determined by two-tailed unpaired Student's t-test. NS, not significant. Data are pooled from 2-3 independent experiments; n=4-15 per group. FIG. 3A: *p=0.0231 (ivag vs. s.c.+pull) and ***p=0.0006 (s.c. vs. s.c.+pull). FIG. 3C: **p=0.0053 (ivag vs. s.c.+pull). FIG. 3D: **p=0.0081 (ivag vs. s.c.), *p=0.0382 (s.c. vs. s.c.+pull), FIG. 3E: *p=0.0490 (ivag vs. s.c.+pull).

FIG. 4, comprising FIGS. 4A through 4E, depicts the results of experiments showing that the prime and pull vaccination regimen protects mice from lethal genital HSV-2 challenge. FIG. 4A: Weight loss in mice immunized as shown in FIG. 1A and then challenged vaginally with a lethal dose of HSV-2 4 weeks post pull. FIG. 4B: Disease severity in mice immunized as shown in FIG. 1A and then challenged vaginally with a lethal dose of HSV-2 four weeks post pull. A higher disease score indicates more severe disease symptoms. FIG. 4C: Survival in mice immunized as shown in FIG. 1A and then challenged vaginally with a lethal dose of HSV-2 4 weeks post pull. FIG. 4D: HSV-2 viral titres from vaginal washes collected at the indicated time points post challenge with HSV-2. Dashed line indicates limit of detection, none detected. n=11 (non-immunized), n=9 (intravaginal immunization), n=12 (subcutaneously immunized control, subcutaneously immunized plus pull). FIG. 4E Viral titres were measured in the dorsal root ganglia 6-7 days post challenge. Number×ND is the number of mice in which no virus was detected. n=6-11 per group. Statistical significance was measured by two-way ANOVA (FIGS. 4A, 4B, and 4D), log-rank (Mantel-Cox) test (FIG. 4C) or two-tailed unpaired Student's t-test (FIG. 4E). Data are pooled from 3-5 independent experiments. FIG. 4A: ***p=0.0006 (s.c. vs. s.c.+pull), *p=0.0425 (ivag vs. s.c.+pull). FIG. 4B: ***p<0.0001 (s.c. vs. s.c.+pull), **p=0.0063 (ivag vs. s.c.+pull). FIG. 4C: **p=0.002 (s.c. vs. s.c.+pull). FIG. 4D: ***p=0.0002 (unimmunized vs. s.c.+pull), ns=not significant (s.c. vs. s.c.+pull). FIG. 4E: ***p=0.0007 (unimmunized vs. s.c.), **p=0.0017 (unimmunized vs. s.c.+pull), and *p=0.0328 (s.c. vs. s.c.+pull).

FIG. 5 depicts the results of experiments showing that HSV-2 antigen is not present in vagina after subcutaneous immunization. Mice were immunized either ivag or s.c. with TK⁻ HSV-2. On day 5 p.i., vaginal tissue from immunized and naive mice was harvested and HSV-2 antigen was measured by gPCR. Data represent two independent experiments. Error bar is SEM.

FIG. 6, comprising FIGS. 6A and 6B, depicts the results of experiments showing that endogenous HSV-specific CD8 T cells are recruited to the vagina by the prime and pull vaccination regimen. Depo-treated naive mice were immunized ivag or s.c. with TK⁻ HSV-2. Five days p.i., s.c. immunized mice were treated ivag with CXCL9 and CXCL10 (s.c.+pull) or PBS (s.c.). One day later, endogenous RSV-specific T cells were enumerated in the indicated tissues. FIG. 6A: gB-specific CD8 T cells in the vagina and spleen were identified by MHCI tetramer. FIG. 6B: CD44⁺ CD4 T cell numbers in the vagina. Statistical significance was measured by unpaired Student's t-test. *p<0.05. **p<0.01 and ns=not significant. n=6-11 per group. Data are pooled from three independent experiments. Error bars show SEM.

FIG. 7 depicts the results of example experiments demonstrating that inflammatory innate cells are not recruited by the prime and pull vaccination regimen. Five days post ivag or s.c. immunization with TK-HSV-2, mice were treated with the chemokine pull. Recruitment of different cell populations were analyzed one day post-pull in the vagina. Naive mice were unimmunized. Difference between s.c. and s.c.+pull is not significant for any cell population by unpaired Student's t-test. n=3 (naive), n 9 (ivag, s.c., s.c.+pull). Data are pooled from three independent experiments. Error bars show SEM.

FIG. 8, comprising FIGS. 8A through 8D, depicts data indicating CD8 T cell recruitment during prime and pull vaccination regimen does not require CD4 T cell help. FIG. 8A: Experimental schematic. Mice were immunized ivag or s.c. with TK-HSV-2. At day 3 and day 5 p.i., s.c. immunized mice were injected intraperitoneally with a CD4 antibody (Ab). At day 5 p.i., chemokine pull was applied vag. T cell numbers were determined one day post-pull. FIG. 8B: CD4 T numbers were determined in the vaginas of depleting antibody-treated and untreated mice. FIG. 8C: Frequency of donor gBT-1 CD8 T cells was measured in the vagina one day post-pull. Plots are gated on total CD8 T cells. Numbers in plots indicate percent of total CD8T cells that are gBT-1. FIG. 8D: Number of donor gBT-1 CD8 T cells was measured in the indicated tissues. n=6 per group. Statistical significance was measured by unpaired Student's t-test*p<0.05, **p<0.01, ns=not significant. Data are pooled from 2 independent experiments. Error bars show SEM.

FIG. 9, comprising FIGS. 9A through 9D, depicts the results of experiments showing that prime and pull vaccination regimen provides protection against lethal WT HSV-2 challenge in the absence of TCR Tg CD8 T cells, Depo-treated mice were immunized either ivag or s.c. with TK⁻ HSV-2. At 5 days p.i., s.c. immunized mice were treated with either the chemokine pull or PBS. Four weeks post-pull, mice were challenge ivag with a lethal dose of WT HSV-2. Weight loss (FIG. 9A), disease scores (FIG. 9B), survival (FIG. 9C), and viral filters (FIG. 9D) were monitored for two weeks post challenge. *P<0.05, **P<01, and ns=not significant, Statistical significance was measured by two-way ANOVA (FIGS. 9A, 9B, and 9D) or log-rank (Mantel-Cox) test (FIG. 9C). n=5 (unimmunized, ivag, s.c.+pull), n=6 (s.c.), Data are pooled from two independent experiments. Error bars show SEM.

FIG. 10 depicts the results of experiments indicating that HSV-specific antibodies in the vagina are not affected by chemokine pull. Vaginal washes from unimmunized, ivag immunized, s.c. immunized and prime and pull vaccination regimen mice were collected at 3-4 weeks post-pull. HSV-specific antibodies were measured by ELISA. Data are pooled from two independent experiments. Error bars show SEM.

FIG. 11, comprising FIGS. 11A through 11D, depicts the results of experiments indicating prime and pull vaccination regimen mice are still protected at least 12 weeks post-pull from WT HSV-2 challenge. 10⁵ gBT-I CD8 T cells were adoptively transferred to Depo-treated recipients and mice were immunized ivag or s.c. with TK-HSV-2 or left unimmunized. Five days p.i., s.c. immunized mice were treated intravaginally with PBS (s.c.) or 3 μg each CXCL9 and CXCL10 All groups were Depo-treated again 1-2 weeks post-pull. At 10-12 weeks post-pull, mice were challenged ivag with WT HSV-2 and monitored for weight loss (FIG. 11A), disease score (FIG. 11B) and survival (FIG. 11C). Viral titers were measured from vaginal washes harvested during the first 5 days of challenge ID). Statistical significance was measured by two-way ANOVA (FIGS. 11A, 11B, and 11D) or log-rank (Mantel-Cox) test (FIG. 11C). *p<0.05, ns=not significant. n=3 (unimmunized), n=7 (ivag, s.c.), n=11 (s.c.+pull). Data are pooled from three independent experiments. Error bars show SEM.

### DETAILED DESCRIPTION

The present invention relates to a vaccination strategy that establishes protective immunity, including a local protective memory T cell population, within a tissue, including immunologically restrictive tissue. By way of one non-limiting example, the genital mucosa, which is a portal of entry for sexually transmitted pathogens, is an immunologically restrictive tissue that prevents entry of activated T cells, unless inflammation or infection is present. To overcome this obstacle, the vaccination strategy of the present invention, also referred to herein as a prime and pull vaccination regimen, is used to establish immunoprotection (including, in part, a local protective memory T cell population) at a potential site of pathogen exposure. The prime and pull vaccination regimen occurs in two phases. In one embodiment, the first phase (i.e., prime) is a parenteral vaccination to elicit a systemic activated T cell response. In one embodiment, the second phase (i.e., pull) is the recruitment of activated T cells to the desired anatomic location (e.g., immunologically restrictive tissue) by means of a local (e.g., topical) chemokine administration, where such recruited T cells establish a persistent residence and thereby mediate protective immunity.

The prime and pull vaccination regimen of the present invention is useful for establishing immunoprotection against cancer or infection involving any desired anatomic location, including immunologically restrictive tissue, such as, by way of non-limiting examples, the genital mucosa, a tumor, the skin, the central nervous system, the peripheral nervous system, the testes, the placenta, the eye, the intestine, and the lung airways. The prime and pull vaccination regimen of the present invention is useful for establishing immunoprotection against a variety of tumors and infectious pathogens including, but not limited to, viruses, bacteria, fungus and protozoa. In addition to establishing immunoprotection against a tumor or a pathogen in a subject before exposure to the tumor or pathogen, the prime and pull vaccination regimen of the invention can be used to treat cancer or infection in a subject that has already developed cancer or has been infected by a pathogen, by recruiting activated T cells to an affected anatomic location, such as, by a way of example, an immunologically restrictive tissue.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The terms “immunogen,” “antigen” or “Ag,” as used herein, is defined as a molecule that induces an immune response. This immune response may involve either antibody production, or the activation of specific immunologically-competent cells, or both. The skilled artisan will understand that any macromolecule, including virtually all proteins or peptides, can serve as an immunogen. Furthermore, immunogens can be derived from recombinant or genomic DNA. A skilled artisan will understand that any DNA, which comprises a nucleotide sequences or a partial nucleotide sequence encoding a protein that elicits an immune response therefore encodes an “immunogen” as that term is used herein. Furthermore, one skilled in the art will understand that an immunogen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an immunogen need not be encoded by a “gene” at all. It is readily apparent that an immunogen can be generated, synthesized or can be derived from a biological sample.

As used herein, by “combination therapy” is meant that a first agent is administered in conjunction with another agent. “In conjunction with” refers to administration of one treatment modality in addition to another treatment modality. As such, “in conjunction with” refers to administration of one treatment modality before, during, or after delivery of the other treatment modality to the individual. Such combinations are considered to be part of a single treatment regimen or regime.

As used herein, the term “concurrent administration” means that the administration of the first therapy and that of a second therapy in a combination therapy overlap with each other.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “expression” as used herein is defined as the transcription and/or translation of a particular nucleotide sequence.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” as used herein, is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “immune response” includes T cell mediated and/or B-cell mediated immune responses. Exemplary immune responses include T cell responses, e.g., cytokine production and cellular cytotoxicity, and B cell responses, e.g., antibody production. In addition, the term immune response includes immune responses that are indirectly affected by T cell activation, e.g., antibody production (humoral responses) and activation of cytokine responsive cells, e.g., macrophages Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

An “immunologically restrictive tissue,” as used herein, is a organ, tissue or area in or on the body of a subject that is poorly accessible by immune effectors, such as circulating memory lymphocytes. Examples of immunologically restrictive tissue include, but are not limited to, the genital mucosa, a tumor, the skin, the central nervous system, the peripheral nervous system, the testes, the placenta, the eye, the intestine, and the lung airways.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Parenteral” administration of a composition includes, e.g., subcutaneous (s.c.), intravenous (i.v.), intramuscular (i.m.), or intradermal (i.d.) injection or infusion techniques.

“Topical” administration of a composition includes contacting a body surface of the subject, including the skin, the eye, or the mucosa, with the composition.

As used herein, “pathogen” refers to a virus, a bacterium, a fungus, or a protozoan associated with disease.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “specifically binds,” is used herein in reference to the interaction of an antibody, a polypeptide, or a peptide with a second chemical species, to mean that the interaction is dependent upon the presence of a particular structure (e.g., an antigenic determinant, binding domain, or epitope) on the chemical species. For example, a ligand recognizes and binds to a specific receptor structure rather than to proteins generally. For example, an antibody recognizes and binds to a specific protein structure rather than to proteins generally.

The term “therapeutic” as used herein means a treatment and/or prophylaxis. A therapeutic effect is obtained by a diminution, suppression, remission, or eradication of a disease state.

The term “therapeutically effective amount” refers to the amount of the subject compound that will elicit the biological or clinical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The terms “transfected” or “transduced” as used herein refers to a process by which exogenous nucleic acid is transferred or introduced into the host cell. A “transfected” or “transduced” cell is one which has been transfected, transduced with exogenous nucleic acid. The cell includes the primary subject cell and its progeny.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to a vaccination strategy that establishes protective immunity, including a local protective memory T cell population, within a tissue, including immunologically restrictive tissue. The vaccination strategy of the present invention, also referred to herein as the prime and pull vaccination regimen, is used to establish immunoprotection (including, in part, a local protective memory T cell population) at a potential site of cancer or pathogen exposure.

The prime and pull vaccination regimen occurs in two phases. For example, the first phase (i.e., prime) is a parenteral vaccination to elicit a systemic activated T cell response. The second phase (i.e., pull) is the recruitment of activated T cells to the desired anatomic location (e g, immunologically restrictive tissue) by means of a local (e.g., topical) chemokine administration, where such recruited T cells establish a persistent residence and thereby mediate protective immunity. In some embodiments, the recruited T cells are CXCR3+CD4+ T cells. In other embodiments, the recruited T cells are CXCR3+CD8+ T cells. In particular embodiments, the recruited T cells are both CXCR3+CD4+ T cells and CXCR3+CD8+ T cells. In some embodiments, the recruited T cells are CCR5+CD4+ T cells. In other embodiments, the recruited T cells are CCR5+CD8+ T cells. In particular embodiments, the recruited T cells are both CCR5+CD4+ T cells and CCR5+CD8+ T cells. In some embodiments, the chemokine is (C—X—C motif) ligand 9 (CXCL9). In other embodiments, the chemokine is CXCL10. In other embodiments, the chemokine is CCL5. In particular embodiments, the chemokine is a combination of at least two of CXCL9, CXCL10 and CCL5. In some embodiments, the two phases of the prime and pull vaccination regimen occur concurrently. In other embodiments, the two phases of the primer and pull vaccination regimen occur in series. In some embodiments, the two phases of the prime and pull vaccination regimen are temporally separate. In other embodiments, the two phases of the prime and pull vaccination regimen temporally overlap.

The prime and pull vaccination regimen of the present invention is useful for establishing immunoprotection against cancer or infection involving any desired anatomic location, including immunologically restrictive tissue, such as, by way of non-limiting examples, the genital mucosa, a tumor, the skin, the central nervous system, the peripheral nervous system, the testes, the placenta, the eye, the intestine, and the lung airways. The prime and pull vaccination regimen of the present invention is useful for establishing immunoprotection against a variety of infectious pathogens including, but not limited to, viruses (e.g., herpes simplex virus (HSV)-1 (HSV-1), HSV-2, human immunodeficiency virus (HIV), human papilloma virus (HPV), etc.), bacteria (e.g., chlamydia, gonorrhea, syphilis, etc.), fungus and protozoa (e.g., trichomonas, etc.). In addition to establishing immunoprotection against cancer or a pathogen in a subject before the development of cancer or exposure to the pathogen, the prime and pull vaccination regimen of the invention can be used to treat cancer or infection in a subject that has already developed cancer or been infected by a pathogen, by recruiting activated T cells to an affected anatomic location, such as, by a way of example, an immunologically restrictive tissue.

### Vaccination

The invention provides an immunogenic composition comprising a polypeptide, or a combination of polypeptides, derived from a tumor or a pathogen and useful in eliciting an immune response. The immunogenic composition comprising one or more polypeptides of the invention is useful not only as a prophylactic therapeutic agent for eliciting immunoprotection, but is also useful as a therapeutic agent for treatment of an ongoing disease or disorder (i.e., infection, cancer, etc.) of a subject.

In one embodiment, the immunogenic composition comprising a polypeptide, or a combination of polypeptides, comprises at least one polypeptide, or fragment thereof, derived from HSV-2. In a particular embodiment, the immunogenic composition comprising a polypeptide, or a combination of polypeptides, comprises HSV glycoprotein B (gB), or fragment thereof.

In one embodiment, the immunogenic composition comprising a polypeptide, or a combination of polypeptides, comprises at least one polypeptide, or fragment thereof, derived from HSV-1. In another embodiment, the immunogenic composition comprising a polypeptide, or a combination of polypeptides, comprises at least one polypeptide, or fragment thereof, derived from HIV-1. In another embodiment, the immunogenic composition comprising a polypeptide, or a combination of polypeptides, comprises at least one polypeptide, or fragment thereof, derived from a tumor cell (i.e., a tumor antigen).

The present invention also provides methods of preventing, inhibiting, and treating cancer or infection. In one embodiment, the vaccination methods of the invention induce protective immunity against cancer or a pathogen, by generating an immune response directed against the cancer or the pathogen. In one embodiment, the methods of the invention induce production of pathogen-specific antibodies. In another embodiment, the methods of the invention induce a pathogen-specific cell-mediated immune response. In another embodiment, the methods of the invention induce production of pathogen-specific antibodies and a pathogen-specific cell-mediated immune response. In one embodiment, the methods of the invention induce production of tumor-specific antibodies. In another embodiment, the methods of the invention induce a tumor-specific cell-mediated immune response. In another embodiment, the methods of the invention induce production of tumor-specific antibodies and a tumor-specific cell-mediated immune response.

The present invention also provides polynucleotides that encode the immunogenic polypeptides described herein. For example, in various embodiments, the composition of the present invention comprises a polynucleotide encoding at least one polypeptide derived from a pathogen. In other various embodiments, the composition of the present invention comprises a polynucleotide encoding at least one polypeptide that is a tumor antigen. In one embodiment, the composition of the present invention comprises a polynucleotide encoding a polypeptide, or fragment thereof, of HSV-2. In a particular embodiment, the polynucleotide encoding a gBT-I polypeptide, or fragment thereof. In another embodiment, the composition of the present invention comprises a polynucleotide encoding a polypeptide, or fragment thereof, of HSV-1. In another embodiment, the composition of the present invention comprises a polynucleotide encoding a polypeptide, or fragment thereof, of HIV-1.

The polynucleotide can be RNA or DNA. In various embodiments, the composition comprises a DNA vaccine. In one embodiment, the methods comprise administering a DNA vaccine to a subject, thereby inducing immunity against cancer or a pathogen. In some embodiments, the method comprises electroporation.

In one embodiment, the immunogenic compositions of the invention are administered parenterally. By way of non-limiting examples, the immunogenic compositions of the invention can be parenterally administered subcutaneously, intramuscularly, or intradermally.

Immunogenic polypeptides useful in the present invention can be prepared using well known techniques. For example, the polypeptides can be isolated from a tumor or from a pathogen, or can be prepared synthetically, using either recombinant DNA technology or chemical synthesis. Polypeptides of the present invention may be synthesized individually or as longer polypeptides composed of two or more polypeptides. The polypeptides of the present invention are preferably isolated, i.e., substantially free of other naturally occurring host cell proteins and fragments thereof. In some embodiments, the immunogenic polypeptides of the invention are a component in a complex mixture, such as a composition comprising a live pathogenic organism, a live attenuated pathogenic organism, an inactivated pathogenic organism, or a dead pathogenic organism.

The immunogenic polypeptides of the present invention may contain modifications, such as glycosylation, aglycosylation, side chain oxidation, or phosphorylation; so long as the modifications do not destroy the biological or immunogenic activity of the polypeptides. Other modifications include incorporation of D-amino acids or other amino acid mimetics that can be used, for example, to increase the serum half-life of the polypeptides.

The immunogenic polypeptides of the invention can be modified whereby the amino acid is substituted for a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). The letters in parentheses indicate the one-letter codes of amino acids.

The immunogenic polypeptides of the invention can be prepared as a combination, which includes two or more of polypeptides of the invention, for use as a prophylactic or therapeutic vaccine for prevention or treatment of cancer or of infection by a pathogen. The immunogenic polypeptides may be in a cocktail or may be conjugated to each other using standard techniques. For example, the immunogenic polypeptides can be expressed as a single polypeptide sequence.

The present invention should also be construed to encompass “mutants,” “derivatives,” and “variants” of the polypeptides of the invention (or of the DNA encoding the same) which mutants, derivatives and variants are polypeptides which are altered in one or more amino acids (or, when referring to the nucleotide sequence encoding the same, are altered in one or more base pairs) such that the resulting polypeptide (or DNA) is not identical to the sequences described herein, but has the same biologic or immunogenic property as the polypeptides disclosed herein.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art, several alternative polynucleotides are possible due to redundant codons, while retaining the biologic or immunogenic activity of the translated polypeptides.

Further, the invention encompasses an isolated nucleic acid encoding a polypeptide having substantial homology to the polypeptides described herein. Preferably, the nucleotide sequence of an isolated nucleic acid encoding a polypeptide of the invention is “substantially homologous,” that is, is about 60% homologous, more preferably about 70% homologous, even more preferably about 80% homologous, more preferably about 90% homologous, even more preferably, about 95% homologous, and even more preferably about 99% homologous to a nucleotide sequence of an isolated nucleic acid encoding a polypeptide of the invention.

It is to be understood explicitly that the scope of the present invention encompasses homologs, analogs, variants, fragments, derivatives and salts, including shorter and longer polypeptides and polynucleotides, as well as polypeptide and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these modifications must preserve the immunologic activity of the original molecule. Specifically any active fragments of the active polypeptides as well as extensions, conjugates and mixtures are included and are disclosed herein according to the principles of the present invention.

The invention should be construed to include any and all isolated nucleic acids which are homologous to the nucleic acids described and referenced herein, provided these homologous nucleic acids encode polypeptides having the biological activity of the polypeptides disclosed herein.

The skilled artisan would understand that the nucleic acids of the invention encompass an RNA or a DNA sequence encoding a polypeptide of the invention, and any modified forms thereof, including chemical modifications of the DNA or RNA which render the nucleotide sequence more stable when it is cell-free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

Further, any number of procedures may be used for the generation of mutant, derivative or variant forms of a polypeptide of the invention using recombinant DNA methodology well known in the art such as, for example, that described in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). Procedures for the introduction of amino acid changes in a polypeptide by altering the DNA sequence encoding the polypeptide are well known in the art and are also described in these, and other, treatises.

The nucleic acids encoding the immunogenic polypeptide or combinations of polypeptides of the invention of the invention can be incorporated into suitable vectors, including but not limited to, plasmids, retroviral and lentiviral vectors. Such vectors are well known in the art and are therefore not described in detail herein.

In one embodiment, the invention includes a nucleic acid sequence encoding one or more polypeptides of the invention operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). The incorporation of a desired polynucleotide into a vector and the choice of vectors is well-known in the art as described in, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

The polynucleotide of the invention can be cloned into a number of types of expression vectors. However, the present invention should not be construed to be limited to any particular expression vector. Instead, the present invention should be construed to encompass a wide plethora of vectors which are readily available and/or well-known in the art. For example, the polynucleotide of the invention can be cloned into a vector including, but not limited to a plasmid, a phagemid, a phage derivative, an animal virus, and a cosmid.

In specific embodiments, an expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired nucleotide sequences of the invention, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). The promoters employed may be constitutive, tissue-specific, inducible, and/or useful under the appropriate conditions to direct high level expression of the introduced DNA segment, such as is advantageous in the large-scale production of recombinant proteins and/or polypeptides. The promoter may be heterologous or endogenous.

One example of a constitutive promoter sequence is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter, where the promoter is active only in a desired tissue. Tissue-specific promoters are well known in the art and include, but are not limited to, the HER-2 promoter and the PSA associated promoter sequences.

In some embodiments, the expression vector is modified to increase the expression of the desired polypeptide. For example, the vector can undergo codon optimization to improve expression in a given mammal. For example, the vector can be codon-optimized for human expression. In another embodiment, the expression vector comprises an effective secretory leader. An exemplary leader is an IgE leader sequence. In another embodiment, the expression vector comprises a Kozak element to initiate translation. In another embodiment, the nucleic acid is removed of cis-acting sequence motifs/RNA secondary structures that would impede translation. Such modifications, and others, are known in the art for use in DNA vaccines (Kutzler et al, 2008, Nat. Rev. Gen. 9: 776-788; PCT App. No. PCT/US2007/000886; PCT App. No.; PCT/US2004/018962).

The present invention provides methods of preventing, inhibiting, and treating cancer and infection in a subject. The methods of the invention provoke an immune response in the subject. In one embodiment, the methods of the invention induce a pathogen-specific humoral immune response. In some embodiments, the pathogen-specific humoral immune response includes the production of pathogen-specific antibodies, including neutralizing antibodies. In another embodiment, the methods of the invention induce a pathogen-specific cell-mediated immune response. In some embodiments, the pathogen-specific humoral immune response includes the induction of pathogen-specific cytotoxic T lymphocytes. In one embodiment, the methods of the invention induce a tumor-specific humoral immune response. In some embodiments, the tumor-specific humoral immune response includes the production of tumor-specific antibodies. In another embodiment, the methods of the invention induce a tumor-specific cell-mediated immune response. In some embodiments, the tumor specific humoral immune response includes the induction of tumor-specific cytotoxic T lymphocytes.

In one embodiment, the methods of the invention comprise administering to a subject, an effective amount of at least one immunogenic polypeptide derived from a tumor or from a pathogen. In one embodiment, the immunogenic polypeptide is delivered to a cell or population of cells. In one embodiment, the immunogenic polypeptide is delivered to the cells in vivo, for example, intramuscularly or subcutaneously. In another embodiment, the immunogenic polypeptide is delivered to the cells ex vivo, where the cells are then administered to the subject. Preferably, the cells also originate from the subject.

In one embodiment, the methods of the invention comprise administering to a subject, an effective amount of a nucleic acid encoding a polypeptide of a tumor or of a pathogen. In one embodiment, the nucleic acid is delivered to a cell or population of cells. In one embodiment, the nucleic acid is delivered to the cells in vivo, for example, intramuscularly or subcutaneously. In another embodiment, the nucleic acid is delivered to the cells ex vivo, where the cells are then administered to the subject. Preferably, the cells also originate from the subject.

In one embodiment, the methods of the invention may be used in an ex vivo vaccination method, where the immunogenic compositions of the invention are delivered to a cell or cell population, which are administered to the subject, thereby inducing an immune response through, by way of example, the generation of tumor-specific antibodies, anti-tumor cell-mediated immune response, pathogen-specific antibodies or an anti-pathogen cell-mediated immune response.

In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

In other embodiments, the polypeptides of the invention are delivered into cells using in vitro transcribed mRNA. In vitro transcribed mRNA can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient. The methods also provide the ability to control the level of expression over a wide range by changing, for example, the promoter or the amount of input RNA, making it possible to individually regulate the expression level. Furthermore, the PCR-based technique of mRNA production greatly facilitates the design of the chimeric receptor mRNAs with different structures and combination of their domains. For example, varying of different intracellular effector/costimulator domains on multiple chimeric receptors in the same cell allows determination of the structure of the receptor combinations which assess the highest level of cytotoxicity against multi-antigenic targets, and at the same time lowest cytotoxicity toward normal cells.

In vitro-transcribed RNA (IVT-RNA) makes use of two different strategies both of which have been successively tested in various animal models. Cells are transfected with in vitro-transcribed RNA by means of lipofection or electroporation. Preferably, it is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the nucleic acid encoding an immunogenic polypeptide can be delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. No. 6,678,556, U.S. Pat. No. 7,171,264, and U.S. Pat. No. 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. No. 6,567,694; U.S. Pat. No. 6,516,223, U.S. Pat. No. 5,993,434, U.S. Pat. No. 6,181,964, U.S. Pat. No. 6,241,701, and U.S. Pat. No. 6,233,482; electroporation may also be used for transfection of cells in vitro as described e.g. in US20070128708. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents a means for delivering an RNA of interest to a target cell.

For an immunogenic composition to be useful as a vaccine, the immunogenic composition must induce an immune response to the immunogen in a cell, tissue or mammal (e.g., a human). Preferably, the vaccine induces a protective immune response in the mammal. As used herein, an “immunogenic composition” may comprise, by way of examples, an antigen (e.g., a polypeptide), a nucleic acid encoding an antigen (e.g., an expression vector), or a cell expressing or presenting an antigen or cellular component. In particular embodiments, the immunogenic composition comprises or encodes all or part of any immunogenic polypeptide described herein, or an immunologically functional equivalent thereof.

In other embodiments, the immunogenic composition is in a mixture that comprises an additional immunostimulatory agent or nucleic acids encoding such an agent Immunostimulatory agents include, but are not limited to, an additional antigen, an immunomodulator, an antigen presenting cell or an adjuvant. In other embodiments, one or more of the additional agent(s) is covalently bonded to the antigen or an immunostimulatory agent, in any combination. In certain embodiments, the immunogenic composition is conjugated to or comprises HLA anchor motif amino acids.

In the context of the present invention, the term “vaccine” refers to a substance that induces anti-cancer or anti-pathogen immunity or suppresses the cancer or the pathogen upon later introduction of the cancer or pathogen into the subject.

A vaccine of the present invention may vary in its composition of nucleic acid, polypeptide, and/or other cellular components. In a non-limiting example, a nucleic acid encoding an immunogenic polypeptide might also be formulated with an adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

In one embodiment, the vaccine of the invention includes, but is not limited to a polypeptide mixed with an adjuvant. In another embodiment, the vaccine of the invention includes, but is not limited to, a polypeptide introduced together with an antigen presenting cell (APC). The most common cells used for the latter type of vaccine are bone marrow and peripheral blood derived dendritic cells, as these cells express costimulatory molecules that help activation of T cells. WO/2000/006723 discloses a cellular vaccine composition which includes an APC presenting tumor associated antigen polypeptides. Presenting the polypeptide can be effected by loading the APC with a polynucleotide (e.g., DNA, RNA) encoding the polypeptide or loading the APC with the polypeptide itself.

Thus, the present invention also encompasses a method of inducing anti-cancer or anti-pathogen immunity using one or more of immunogenic polypeptides, or variants thereof. When a particular polypeptide or combination of polypeptides induces an anti-cancer or anti-pathogen immune response upon inoculation into an animal, the polypeptide or combination of polypeptides are determined to have an anti-cancer or anti-pathogen immunity inducing effect. The induction of the anti-pathogen immunity by a polypeptide or combination of polypeptides can be detected by observing in vivo or in vitro the response of the immune system of the host against the polypeptide.

For example, a method for detecting the induction of cytotoxic T lymphocytes is well known. A foreign substance that enters the living body is presented to T cells and B cells by the action of APCs. T cells that respond to the antigen presented by APC in an antigen-specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by a certain polypeptide or combination of polypeptides of the invention can be evaluated by presenting the polypeptide to a T cell by APC, and detecting the induction of CTL. Furthermore, APCs have the effect of activating CD4+ T cells, CD8+ T cells, macrophages, eosinophils and NK cells.

A method for evaluating the inducing action of CTL using dendritic cells (DCs) as APC is well known in the art. DCs are a representative APC having the strongest CTL inducing action among APCs. In this method, the polypeptide, or combination of polypeptides, is initially contacted with DC and then this DC is contacted with T cells. Detection of T cells having cytotoxic effects against the cells of interest after the contact with DC shows that the polypeptide, or combination of polypeptides, has an activity of inducing the cytotoxic T cells. Furthermore, the induced immune response can be also examined by measuring cytokines produced and released by T helper of CTL in the presence of antigen-presenting cells that carry immobilized polypeptide, or combination of polypeptides, by visualizing using anti-cytokine antibodies, such as an ELISPOT assay.

Apart from DC, peripheral blood mononuclear cells (PBMCs) may also be used as the APC. The induction of CTL is reported to be enhanced by culturing PBMC in the presence of certain cytokines, including for example, GM-CSF and IL-4. Similarly, CTL has been shown to be induced by culturing PBMC in the presence of keyhole limpet hemocyanin (KLH) and IL-7.

The induction of anti-cancer or anti-pathogen immunity by a polypeptide, or combination of polypeptides, can be further confirmed by observing the induction of antibody production against specific immunogens. For example, when antibodies against a polypeptide, or combination of polypeptides, are induced in a subject immunized with the polypeptide, or combination of polypeptides, and when pathology is suppressed by those antibodies, the polypeptide, or combination of polypeptides, are determined to induce anti-cancer or anti-pathogen immunity.

Anti-cancer and anti-pathogen immunity can be induced by administering a vaccine of the invention, and the induction of anti-cancer or anti-pathogen immunity enables treatment and prevention of a disease associated with cancer or the presence of the pathogen. A decrease in mortality of individuals having a disease, a decrease of the disease markers in the blood, alleviation of detectable symptoms accompanying the disease and such are also included in the therapy or prevention of the disease associated with cancer or infection by the pathogen. Such therapeutic and preventive effects are preferably statistically significant, for example, observed at a significance level of 5% or less, wherein the therapeutic or preventive effect of a vaccine against a disease, is compared to a control without vaccine administration. For example, Student's t-test, the Mann-Whitney U-test or ANOVA may be used for determining statistical significance.

The invention provides a method for treating, or preventing, a disease or condition associated cancer or infection by a pathogen. The vaccines and methods of vaccine administration of the invention may be administered prophylactically or therapeutically to subjects suffering from, or at risk of, or susceptible to, developing the disease or condition, including cancer or infection by a pathogen. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The polypeptide, or combination of polypeptides, of the invention having immunological activity, or a polynucleotide or vector encoding such a polypeptide or combination of polypeptides, may be combined with an adjuvant. An adjuvant refers to a compound that enhances the immune response against the polypeptide or combination of polypeptides when administered together (or successively) with the polypeptide having immunological activity. Examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

In various embodiments, the methods of the present invention comprise parenterally administering a composition comprising an immunogenic polypeptide, or a polynucleotide encoding an immunogenic polypeptide, directly to a subject. Administration of the composition can comprise, for example, intramuscular, intravenous, peritoneal, subcutaneous, and intradermal. In one embodiment, delivery of the composition is aided by in vivo electroporation.

Furthermore, the actual dose and schedule can vary depending on whether the compositions are administered in combination with other pharmaceutical compositions, or depending on inter-individual differences in pharmacokinetics, drug disposition, and metabolism. One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the compositions can be further approximated through analogy to compounds known to exert the desired effect.

Administration of the immunogenic composition in accordance with the present invention may be continuous or intermittent, depending, for example, upon the recipient's physiological condition, whether the purpose of the administration is therapeutic or prophylactic, and other factors known to skilled practitioners. The administration of the immunogenic compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

When the immunogenic compositions of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” carrier, diluent, excipient, and/or salt is one that is compatible with the other ingredients of the formulation, and not deleterious to the recipient thereof. The active ingredient for administration may be present as a powder, as granules, as a solution, as a suspension or as an emulsion.

Pharmaceutical formulations containing the immunogenic compositions of the invention can be prepared by procedures known in the art using well known and readily available ingredients. The compositions of the invention can also be formulated as solutions appropriate for parenteral administration, for instance by intramuscular, subcutaneous, intradermal or intravenous routes.

The pharmaceutical formulations of the compositions of the invention can also take the form of an aqueous or anhydrous solution or dispersion, or alternatively the form of an emulsion or suspension.

Thus, the composition may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, polynucleotides, polypeptides and chemokines of this invention can be formulated and administered to treat a variety of disease states (e.g., infection, cancer, etc.) by any means that produces contact of the active agent with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention (e.g., polypeptides, polynucleotides, etc.) may be formulated to be suspended in a pharmaceutically acceptable composition suitable for use in mammals and in particular, in humans. Such formulations include the use of adjuvants such as muramyl dipolypeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Recruitment

Following the establishment of an immune response directed against an immunogen in the subject, the methods of invention include the recruitment of the immune response to a desired anatomic location in the subject. In some embodiments, the desired anatomic location to which the immune response is recruited is an immunologically restricted tissue. Non-limiting examples of immunologically restricted tissue include the genital mucosa, a tumor, the skin, the central nervous system, the peripheral nervous system, the testes, the placenta, the eye, the intestine, and the lung airways.

In some embodiments, the established immune response directed against an immunogen in the subject, includes activated T cells. In one embodiment, the activated T cells are recruited to a desired anatomic location. In some embodiments, the recruited activated T cells are CD4+ T cells. In other embodiments, the recruited activated T cells are CD8+ T cells. In particular embodiments, the recruited activated T cells are both CD4+ T cells and CD8+ T cells.

In one embodiment, the recruited activated T cells are CXCR3+ T cells. In some embodiments, the recruited activated T cells are CXCR3+CD4+ T cells. In other embodiments, the recruited activated T cells are CXCR3+CD8+ T cells. In particular embodiments, the recruited activated T cells are both CXCR3+CD4+ T cells and CXCR3+CD8+ T cells. In one embodiment, the recruited activated T cells are CCR5+ T cells. In some embodiments, the recruited activated T cells are CCR5+CD4+ T cells. In other embodiments, the recruited activated T cells are CCR5+CD8+ T cells. In particular embodiments, the recruited activated T cells are both CCR5+CD4+ T cells and CCR5+CD8+ T cells.

In one embodiment, the recruited activated T cells differentiate into memory T cells. In some embodiments, the memory T cells persist in the subject for an extended period of time. In some embodiments, the memory T cells persist in the subject for an extended period of time in the anatomic location to which they were recruited. In some embodiments, the memory T cells persist in the anatomic location to which they were recruited for at least 2 weeks, at least 1 month, at least 2 months, at least 3 months, at least 4 months, at least 5 months, at least 6 months, at least 1 year, at least 2 years, at least 3 years, at least 4 years, or at least 5 years.

In some embodiments, the T cells are recruited to the desired anatomic location by the local administration of a chemotactic cytokine (i.e., chemokine) at the desired anatomic location. In some embodiments, the chemokine is CXCL9. In other embodiments, the chemokine is CXCL10. In other embodiments, the chemokine is CCL5. In particular embodiments, the chemokine is a combination of at least two of CXCL9, CXCL10 and CCL5.

In some embodiments, the two phases of the prime and pull vaccination regimen occur concurrently, while in other embodiments the two phases occur in series. In some embodiments, the two phases of the prime and pull vaccination regimen are temporally separate, while in other embodiments, the two phases temporally overlap.

Furthermore, a chemokine of the invention may be combined appropriately with a pharmaceutically acceptable carrier. Examples of such carriers are sterilized water, physiological saline, phosphate buffer, culture fluid and such. Furthermore, the chemokine may contain as necessary, stabilizers, suspensions, preservatives, surfactants and such. The chemokine be administered in a single administration or by multiple administrations.

In various embodiments, the methods of the present invention comprise locally administering a composition comprising chemokine directly to a subject. Administration of the composition can be, for example, topical, intramuscular, intradermal, intratumoral, intracranial, or subcutaneous.

Furthermore, the actual dose and schedule can vary depending on whether the compositions are administered in combination with other pharmaceutical compositions, or depending on inter-individual differences in pharmacokinetics, drug disposition, and metabolism. One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the compositions can be further approximated through analogy to compounds known to exert the desired effect.

Administration of the chemokine in accordance with the present invention may be continuous or intermittent, depending, for example, upon the recipient's physiological condition, whether the purpose of the administration is therapeutic or prophylactic, and other factors known to skilled practitioners. The administration of the chemokine of the invention may be essentially continuous over a preselected period of time or may be in a series of spaced doses. The amount administered will vary depending on various factors including, but not limited to, the composition chosen, the particular disease, the weight, the physical condition, the targeted anatomic location, and the age of the subject, and whether prevention or treatment is to be achieved. Such factors can be readily determined by the clinician employing animal models or other test systems which are well known to the art.

When the chemokine compositions of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” carrier, diluent, excipient, and/or salt is one that is compatible with the other ingredients of the formulation, and not deleterious to the recipient thereof. The active ingredient for administration may be present as a powder, as granules, as a solution, as a suspension or as an emulsion.

Pharmaceutical formulations containing the chemokine compositions of the invention can be prepared by procedures known in the art using well known and readily available ingredients. The compositions of the invention can also be formulated as solutions appropriate for administration, for instance by topical, intramuscular, subcutaneous, intradermal, intracranial or intratumoral routes.

The pharmaceutical formulations of the compositions of the invention can also take the form of an aqueous or anhydrous solution or dispersion, or alternatively the form of an emulsion or suspension.

Thus, the composition may be formulated for administration and may be presented in unit dose form in ampules, pre-filled syringes, small volume infusion containers or in multi-dose containers with an added preservative. The active ingredients may take such forms as suspensions, solutions, creams, gels, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of chemokine contained in an individual dose of each dosage form need not in itself constitute an effective amount for since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers. Solutions for administration contain the chemokine, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, solutions can contain preservatives such as benzalkonium chloride, methyl- or propyl-paraben and chlorobutanol. Suitable pharmaceutical carriers are described in Remington's Pharmaceutical Sciences, a standard reference text in this field.

The chemokines of the invention may be formulated to be suspended in a pharmaceutically acceptable composition suitable for use in mammals and in particular, in humans. Such formulations include the use of adjuvants such as muramyl dipolypeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### A Vaccine Strategy that Protects Against Genital Herpes by Establishing Local Memory T Cells

Cellular immunity is critical in mediating protection against viral STIs such as HSV-2 and HIV-1 (Shin Iwasaki, 2012, Nature, 491:463-468). Both viruses enter through the genital mucosa, begin local replication and then spread to other tissues. Although the methods and results described herein highlight the role of the prime and pull vaccination regimen in controlling viral spread to the peripheral nervous system, the usefulness and immunoprotection provided by the prime and pull vaccination regimen is not restricted to neurotropic viruses. HIV-1 enters the genital mucosa and invades the draining lymph node, from which systemic dissemination of the virus occurs (Iwasaki, 2010, Nature Rev. Immunol. 10:699-711). In its current form, the prime and pull vaccination regimen establishes tissue-resident memory CD8⁺ T cells, but not CD4⁺ T cells. Given that a single HIV-1 virion can establish infection in humans (Iwasaki, 2010, Nature Rev. Immunol. 10:699-711), local memory CD8⁺ T cells may be key to protection against HIV-1 (Iwasaki, 2010, Nature Rev. Immunol. 10:699-711) by reducing replication and dissemination of the founder virus, while the absence of local CD4⁺ T cells could limit the availability of immediate target cells. Beyond viral infections, the prime and pull vaccination regimen could be applied to improve recruitment of immune cells to other restrictive microenvironments, such as solid tumors. Effective immunotherapy can be hindered by either decreased or inappropriate expression of chemokines at the tumor tissue, leading to minimal migration of immune cells (Gajewski, 2011, Curr. Opin. Immunol. 23:286-292). Thus, the delivery of appropriate chemokines to the tumor tissue after immunization serves to enhance recruitment of tumor-specific T cells and augment the efficacy of immunotherapies.

Although the methods described herein pair the pull with a subcutaneous immunization (prime), the prime and pull vaccination regimen could be used in conjunction with any priming immunization (Koelle & Corey, 2008, Annu. Rev. Med. 59:381-395) to enhance protection. The ability to boost recruitment of T cells and establish resident T cell populations in immunologically restrictive tissues aids not only in the prevention but also in the treatment of a wide variety of diseases.

The methods and materials of this experimental example are now described.

### Adoptive Transfers, Infections and T Cell Depletion

CD8⁺ T cells (10⁵) from the spleens of naive CD45.1⁺ gBT-I TCR transgenic mice (Mueller et al., 2002, Immunol. Cell Biol. 80:156-163) were adoptively transferred into Depo-Provera (“Depo”; GE Healthcare) treated (Parr et al., 1994, Lab. Invest. 70:369-380), naive 6-week-old C57BL/6 recipients (National Cancer Institute). Recipients were immunized intravaginally or subcutaneously in the neck ruff with 10⁵ or 10⁶ plaque forming units (PFU) of 186TKΔkpn HSV-2 (TK⁺ HSV-2) (Jones et al., 2000, Virology 278:137-150), respectively. Some mice were treated twice with 200 μg anti-CD4 antibody (GK1.5) intraperitoneally to deplete CD4⁺ T cells. Five days post-immunization, subcutaneously immunized mice were vaginally swabbed with a Calginate swab (Fisher) and either PBS or a solution of CXCL9 and CXCL10 (3 μg each, Peprotech) in PBS was delivered via pipette tip into the vagina. For 4-week challenges, mice were infected intravaginally with 5,000 PFU of wild-type HSV-2 186 syn+ (Spang, 1983, J. Virol. 45:332-342). For 10-12 week challenges, mice were treated again with Depo-Provera 9-10 weeks before challenge.

### Mice

Female 6-week-old C57BL/6 mice were purchased from the National Cancer Institute. gBT-I T cell antigen receptor (TCR) transgenic mice specific for the glycoprotein B epitope gB (498-505) were provided by F. R. Carbone and W. R. Heath and bred to C57BL/6-Ly5.2Cr mice (CD45.1⁺) (National Cancer Institute). All procedures used in this study complied with federal and institutional policies of animal care and use.

### Adoptive Transfers and Infections

Spleens were collected from naive CD45.1⁺ gBT-I TCRtransgenic mice and CD8⁺ T cells were magnetically purified by CD8α microbeads or CD8α+ T cell isolation kits (Miltenyi Biotec). Donor cells (10⁵) gBT-I CD8⁺ T cells were adoptively transferred into Depo-Provera-treated (GE Healthcare), 7-8-week-old C57BL/6 recipients retro-orbitally. Mice were then immunized intravaginally or subcutaneously with 10⁵ or 10⁶ plaque forming units (PFU) of 186TKΔkpn HSV-2 (TK⁻ HSV-2) respectively. At 5 days post-infection, the vaginal cavity of mice was swabbed with a Calginate swab (Fisher) and either PBS or a solution of CXCL9 and CXCL10 (3 ng each, Peprotech) in PBS was delivered via pipette tip into the vagina. Where indicated, C57BL/6 mice that did not receive gBT-I cells were primed and pulled in a similar manner. Some subcutaneously immunized mice were intraperitoneally injected with 200 ng anti-CD4 (GK1.5) antibody at day 3 and 5 post infection to deplete CD4⁺ T cells. For the 4-week challenge, non-immunized or previously immunized mice at the indicated time points were infected intravaginally with 5,000 PFU wild-type HSV-2 186 syn⁺. Challenges given at 10-12 weeks post pull were treated with Depo-Provera for a second time 1-2 weeks post pull (9-10 weeks before challenge) before infection with 5,000 PFU wild-type HSV-2 186 syn+.

### Flow Cytometry

At various time points, single cell suspensions from the spleen, lungs, vagina and iliac lymph nodes were prepared for analysis as described (Iijima et al., 2008, J. Exp. Med. 205:3041-3052). Briefly, lungs were digested with collagenase D (Roche). Vaginas were treated with Dispase II (Roche) for 15 min and then collagenase D for 30 min. Cells from the spleen and iliac lymph node were counted by haemocytometer. Lung and vagina cell numbers were quantified using CountBright absolute counting beads (Invitrogen). Dead cells were excluded from analysis using the LIVE/DEAD Fixable Aqua Dead Cell stain kit (Invitrogen). All samples were acquired on an LSRII equipped with a 532-nm green laser (BD Biosciences). All data were analysed with FlowJo (Treestar).

### Antibodies

The following antibodies were used for this study: CD3 (17A2), CD8 (53-6.7), CD44 (1M7), CD45.1 (A20), CXCR3 (CXCR3-173), CD11c (N418), CD11b (M1/70), MHC class II (M5/114.15.2), Ly6G (1A8), F4/80 (BM8), B220 (RA3-6B2), CD19 (ebio1D3) and NK1.1 (PD136) (Biolegend); Ly6C (AL-21)(BD Biosciences); CD4 (RM4-4) (eBioscience); and CD4 (RM4-5) (Biolegend and Invitrogen). H-2K^{b}-gB₄₉₈₋₅₀₅ tetramer was obtained from the National Institutes of Health tetramer core facility.

### Measurement of Viral Titres, Weight and Disease Scores

Vaginal secretions were collected 5 days post challenge using PBS and Calginate swabs. Lumbar and sacral dorsal root ganglia (DRG) were collected at days 6-7 post challenge as described (Malin et al., 2007, Nature Protocols 2:152-160). DRG were homogenized using a motorized pestle (VWR). Titres from vaginal and DRG samples were measured on Vero cell monolayers as previously described (Iijima et al., 2008, J. Exp. Med. 205:3041-3052). Weight loss was measured daily and normalized to body weight on day 0 of challenge. Disease was monitored daily and scored as follows: (0) no disease; (1) genital inflammation; (2) genital lesions and hair loss; (3) hunched posture and ruffled fur; (4) hind limb paralysis; and (5) premoribund (Morrison et al., 1998, Virology 243:178-187). Mice were euthanized before reaching the moribund state due to humane concerns.

### Detection of HSV-2 Antigen by Quantitative PCR

Mice were immunized subcutaneously or intravaginally and were killed at day 5 post infection. Vaginal tissue was collected and genomic DNA was extracted as previously described (Aljanabi & Martinez, 1997, Nucleic Acids Res. 25:4692-4693). Briefly, tissue was homogenized in a salt homogenizing buffer using a motorized pestle. Proteinase K and SDS were added to samples and incubated overnight at 55° C. After addition of a sodium chloride solution, samples were centrifuged and supernatants were transferred to new tubes. Isopropanol was added to the supernatants and incubated at 20° C. for 1 h. DNA was pelleted by centrifugation, washed with ethanol and resuspended in H₂O. HSV-2 was measured with primers detecting glycoprotein B (gB) (Forward: 5′-AGACCAGGGCCGCTGATC-3′ (SEQ ID NO:1); reverse: 5′-GCGCTGGACCTCCGTGTAG-3′ (SEQ ID NO:2) with quantitative polymerase chain reaction (Stratagene). DNA purified from TK⁻ HSV-2 was used as standard to calculate PFU equivalents.

### Measurement of HSV-Specific Antibody Titres

Vaginal secretions were collected from mice with PBS and Calginate swabs 4 weeks post pull. HSV-specific immunoglobulin-G (IgG) was measured by ELISA assay as previously described (Soderberg, 2004, J. Immunol. 173:1908-1913). Known quantities of anti-HSV gB monoclonal antibody (SS10 mouse IgG) was used as a standard.

The results of this experimental example are now described.

After genital HSV-2 infection, chemokine (C—X—C motif) ligand 9 (CXCL9) and CXCL10 expression is induced by interferon-γ secreted by CD4⁺ T cells and mediates recruitment of effector CD8⁺ T cells to the infected tissue via the chemokine receptor CXCR3 (Nakanishi et al., 2009, Nature 462:510-513). CXCR3 is expressed by effector T-helper 1 (T_{H}1) cells, activated CD8⁺ T cells, as well as other cell types (Groom & Luster, 2011, Immunol. Cell Biol. 89:207-215). Thus, to test the hypothesis that the topical application of chemokines CXCL9 and CXCL10 would recruit effector T cells to the vagina in the absence of infection, T cell antigen receptor transgenic CD8⁺ T cells that recognize an epitope within the HSV glycoprotein B (gBT-I) (Mueller et al., 2002, Immunol. Cell Biol. 80:156-163) were used to track the HSV-2 specific CD8 T cell population.

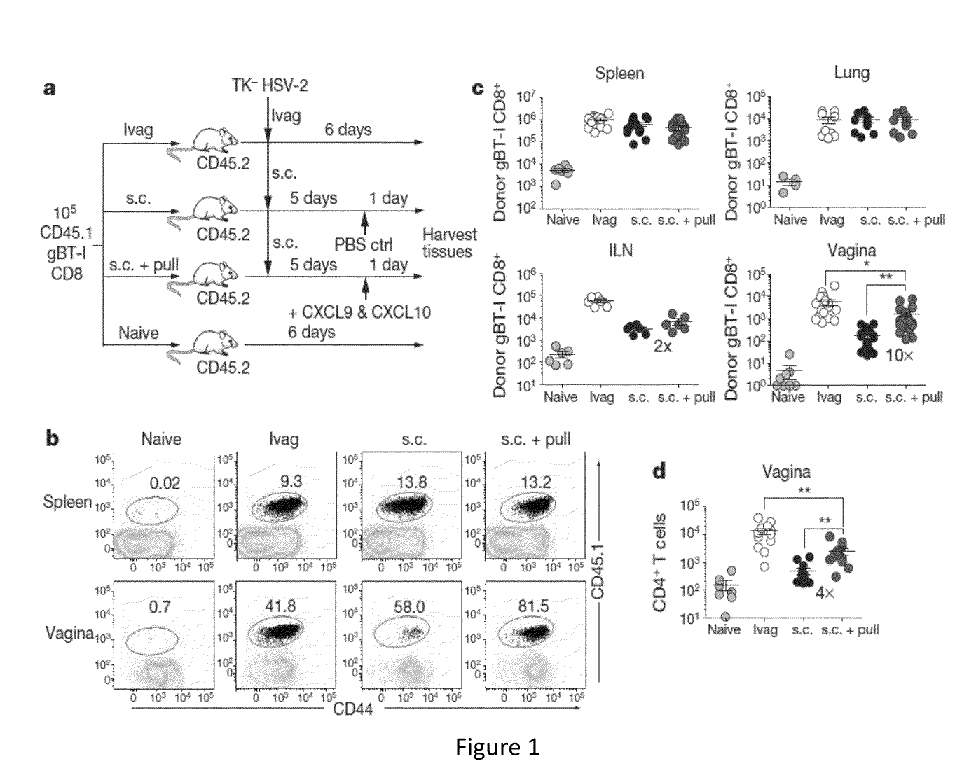

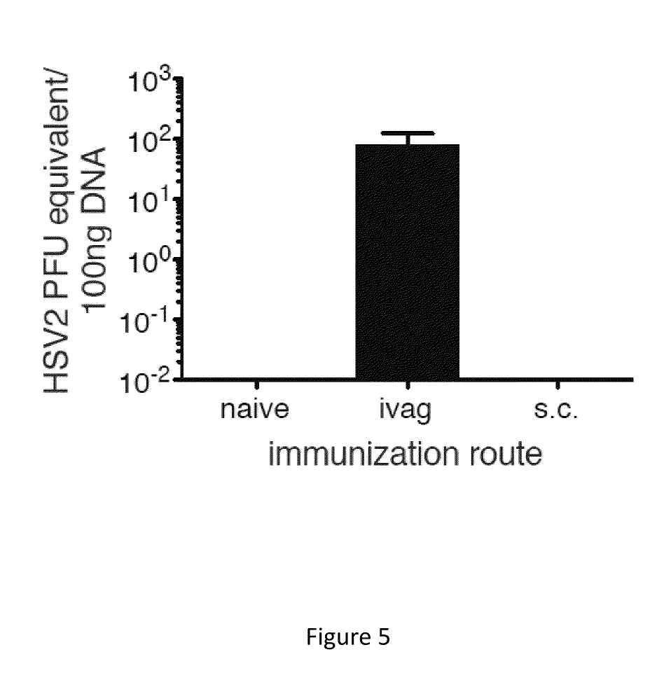

Naive female C57BL/6 mice were transplanted with 10⁵ congenically marked gBT-I CD8⁺ T cells and immunized subcutaneously with an attenuated strain of HSV-2 that lacks thymidine kinase (TK⁻ HSV-2) (Jones et al., 2000, Virology 278:137-150) (FIG. 1A). As expected, this route of immunization resulted in minimal migration of activated CD8⁺ T cells into the vagina (FIG. 1B, 1C). To recruit or ‘pull’ activated HSV-specific CD8⁺ T cells, the chemokines CXCL9 and CXCL10 were topically applied to the vaginal cavity of subcutaneously immunized mice (FIG. 1A). Another group of mice was immunized intravaginally with TK⁺ HSV-2, which served as a positive control for maximal CD8⁺ T cell recruitment to the vagina (FIG. 1B, 1C). At day 6 post infection, all three treatment groups exhibited primary CD8⁺ T cell responses of similar magnitudes, as indicated by the numbers and percentages of systemic gBT-I CD8⁺ T cells found in the spleen (FIG. 1B, 1C). However, the number and percentage of gBT-I CD8⁺ T cells in the vagina were significantly higher in mice treated with the chemokine pull (subcutaneous immunization plus pull) compared to the control subcutaneously immunized mice (FIG. 1B, 1C). Furthermore, the action of the chemokine pull was restricted to the genital mucosa, as gBT-I CD8⁺ T cell recruitment to the vagina-draining iliac lymph nodes was limited (FIG. 1C). Activated CD4⁺ T cells were also strongly recruited to the vagina by the chemokine pull (FIG. 1D). Antigen in the vagina was not responsible for the recruitment, as HSV-2 genomic DNA was absent from the genital tract after subcutaneous immunization (FIG. 5).

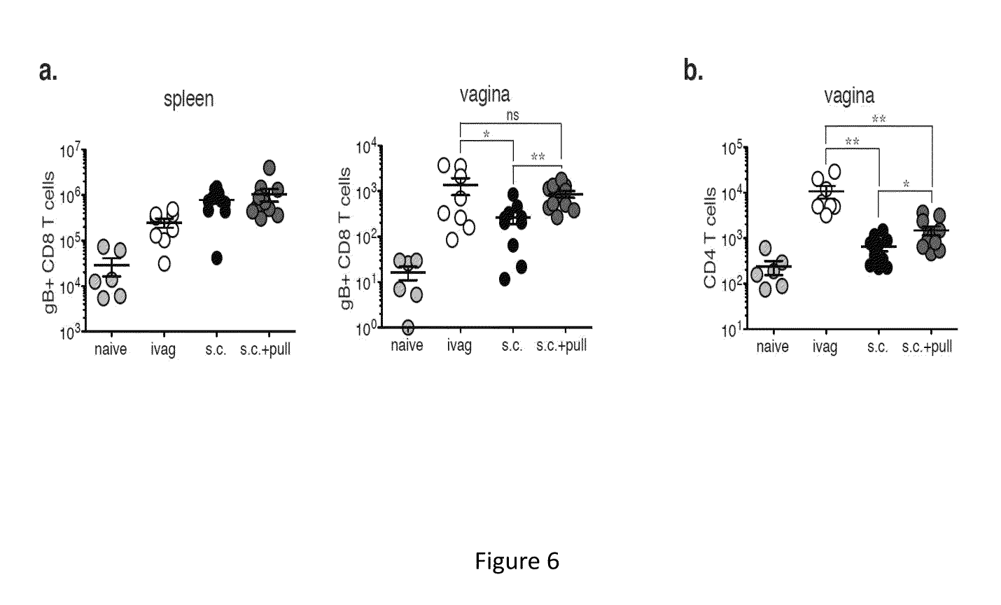

To mimic a vaccination scenario more closely, the potential to recruit endogenous virus-specific T cells by prime and pull vaccination regimen was also tested. Like gBT-I CD8⁺ T cells, the systemic endogenous HSV-specific CD8⁺ T cell response was similar in all immunized groups (FIG. 6A). However, significantly greater numbers of HSV-specific CD8⁺ T cells as well as CD4⁺ T cells were present in the genital tracts of mice treated with the chemokine pull as compared to subcutaneous immunization alone (FIG. 6). Thus, these data show that the prime and pull vaccination regimen is capable of recruiting a large number of parenterally primed T cells to the genital tract with a single topical application of chemokines.

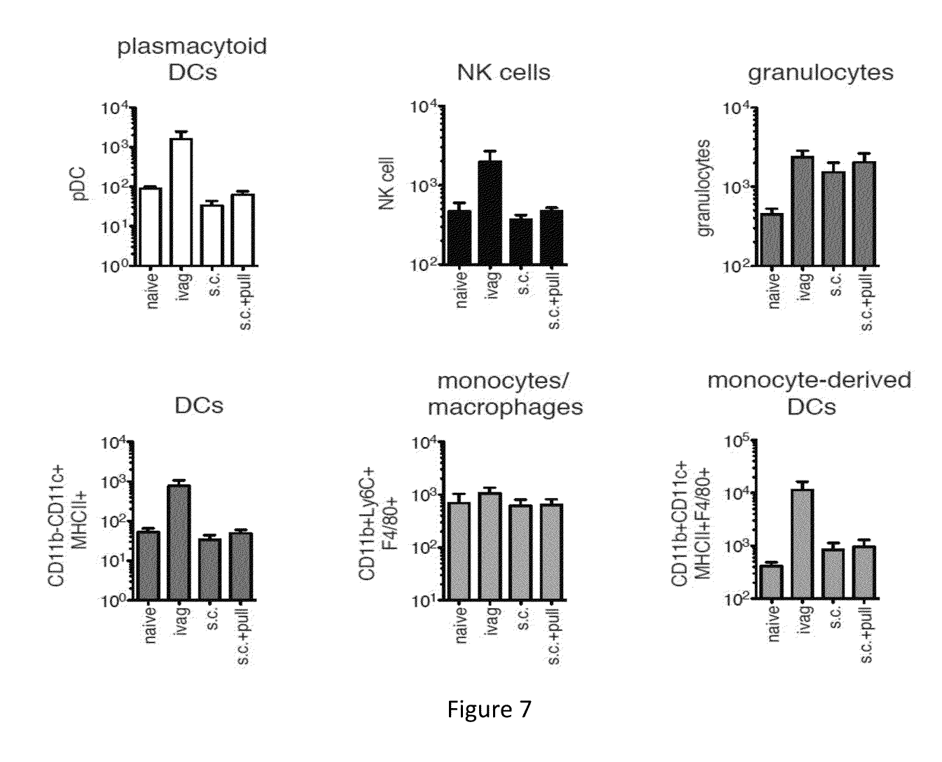

To assess the possible inflammatory consequences of topical chemokine application to the vagina, the presence of innate inflammatory cells after the chemokine pull were examined Other cell types, including natural killer cells and plasmacytoid dendritic cells (pDCs) express CXCR3 (Groom & Luster, 2011, Immunol. Cell Biol. 89:207-215). However, no significant increase in the number of pDCs, natural killer cells, granulocytes, dendritic cells, monocytes, macrophages and monocyte-derived dendritic cells was elicited by the chemokine treatment (subcutaneously immunized plus pull) compared to the subcutaneously immunized control (FIG. 7). These data are consistent with the explanation that topical chemokines do not induce appreciable recruitment of natural killer cells or pDCs to the vagina and that effector T cells are selectively recruited during by the prime and pull vaccination regimen without inducing a general inflammatory response.

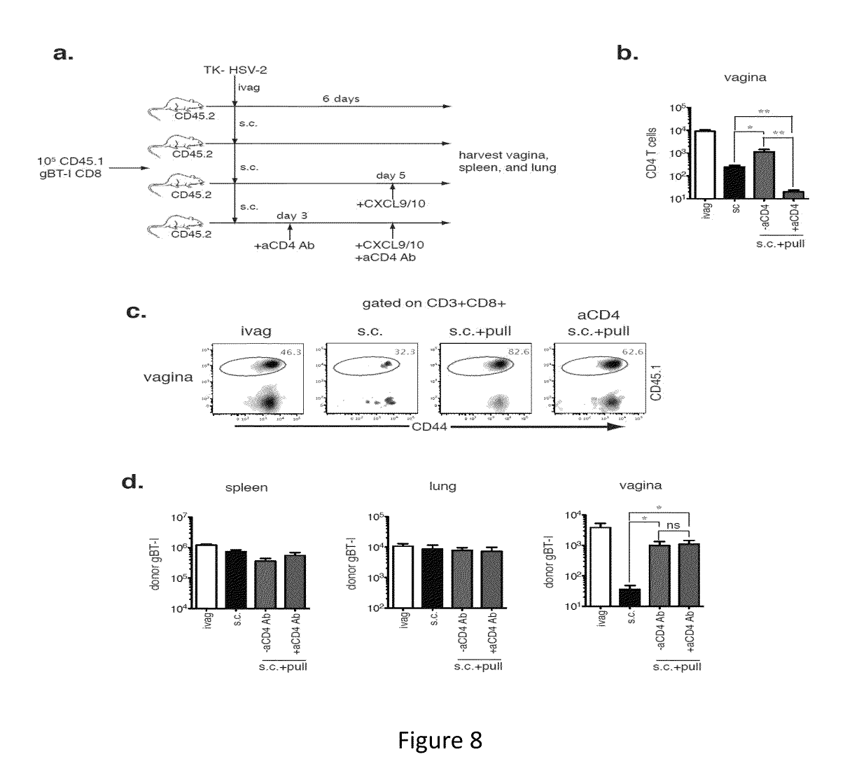

During genital HSV infection, CD4⁺ T cells act as a pioneering population for the migration of virus-specific CD8⁺ T cells by inducing the production of critical chemokines within the tissue (Nakanishi et al., 2009, Nature 462:510-513). To determine whether the recruitment of gBT-I CD8⁺ T cells to the genital tract was similarly dependent on CD4⁺ T cell help during the prime and pull vaccination regimen, subcutaneously immunized mice were injected with a CD4-depleting antibody on day 3 and day 5 post infection to preserve normal CD8⁺ T cell priming (Smith et al., 2004, Nature Immunol. 5:1143-1148), and then treated with the chemokine pull (FIG. 8A). In CD4⁺ T cell-depleted mice (FIG. 8B), both systemic gBT-ICD8⁺ T cell numbers and migration to the vagina were unaffected (FIG. 8C, 8D), indicating that recruitment of effector CD8⁺ T cells to the vagina after chemokine treatment bypasses the requirement for CD4⁺ T cell help.

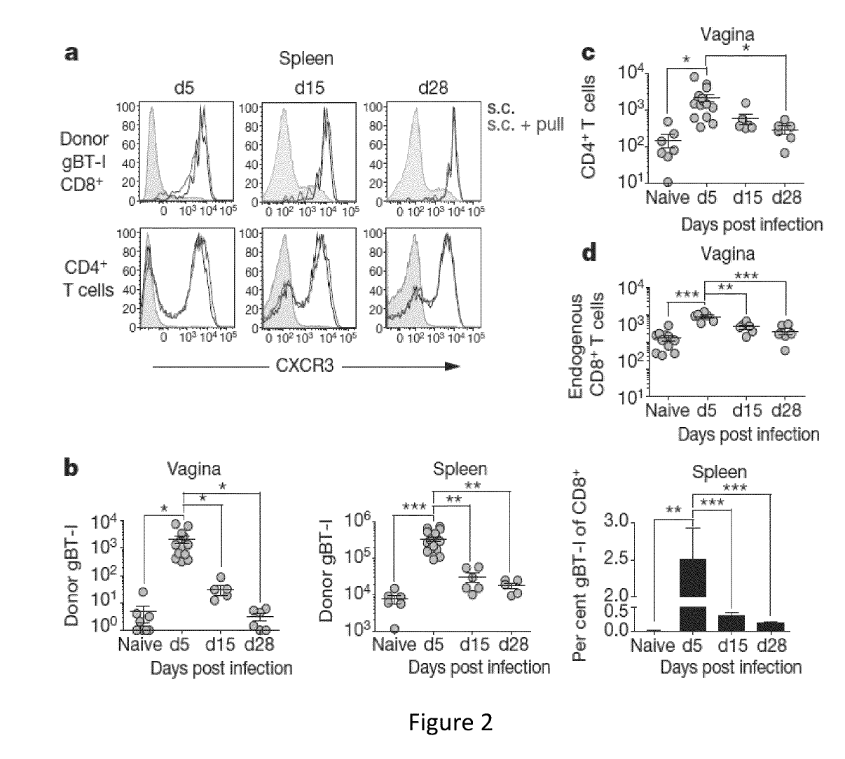

CXCR3 is upregulated on T cells upon activation and remains high through the effector and memory stages (Groom & Luster, 2011, Immunol. Cell Biol. 89:207-215). Having demonstrated that CXCL9 and CXCL10 could recruit CXCR3⁺ effector T cells to the vagina, the efficacy of the chemokine pull at different stages of T cell priming was examined. After subcutaneous TK⁻ HSV-2 immunization, CXCR3 was upregulated on both gBT-I CD8⁺ T cells and CD4⁺ T cells throughout the response (FIG. 2A), suggesting that both effector and memory T cells should be capable of responding to the chemokine pull.

Previous reports have shown that early effector CD8⁺ T cells had an increased ability to migrate to peripheral tissues (Masopust et al., 2010, J. Exp. Med. 207:553-564), so it was next determined whether the timing of chemokine pull dictated the efficacy of T cell recruitment to the genital tract. When subcutaneously immunized mice were treated with the chemokine pull at the effector (day 5), contraction (day 15) and memory (day 28) phase of the T cell response (Kaech & Wherry, 2007, Immunity 27:393-405), it was found that the chemokine pull was most effective at recruiting antigen-specific CD8⁺ T cells during the effector (day 5) phase, which correlated with the increased number and percentage of systemic gBT-ICD8⁺ T cells (FIG. 2B). Despite similar CXCR3 expression (FIG. 2A), memory gBT-I CD8⁺ T cells were not present in the tissue after pull when treated during the memory phase (day 28) (FIG. 2B). While not wishing to be bound by any particular theory, it is believed that this result might be due to altered homing patterns (Masopust et al., 2010, J. Exp. Med. 207:553-564; Weninger et al., 2001, J. Exp. Med. 194:953-966) and the reduced number and percentage of gBT-I CD8⁺ T cells in circulation at the memory time point. Recruitment of CD44⁺CD4⁺ T cells (FIG. 2C) and endogenous CD8⁺ T cells (FIG. 2D) followed a similar pattern. Collectively, these data indicate that the chemokine pull is most effective at recruiting recently activated effector CD8⁺ T cells that are circulating at high frequency, establishing a specific time frame within which the chemokine pull should be administered after priming.

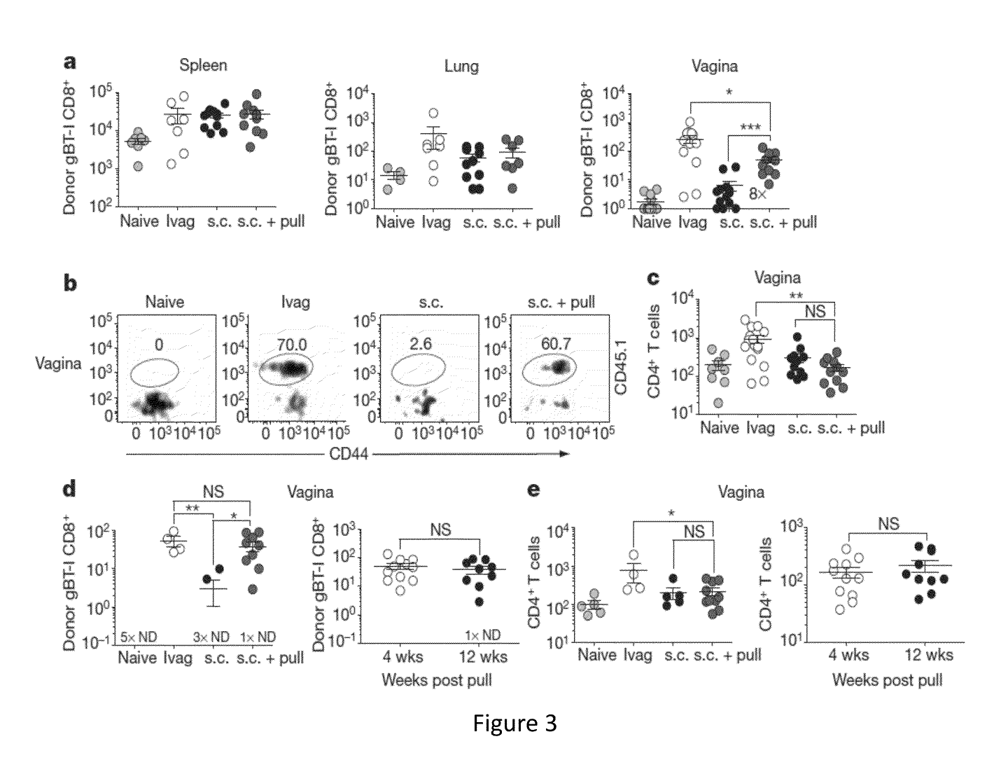

Without wishing to be bound by any particular theory, it is believed that for the prime and pull vaccination regimen to be an optimally effective vaccination strategy, pathogen-specific T cells must be retained within the tissue for an extended time and establish a pool of memory cells. To determine whether the effector gBT-I CD8⁺ T cells recruited into the vagina after the prime and pull vaccination regimen were capable of establishing a long-term population of memory CD8 T cells, the presence of gBT-I CD8⁺ T cells 4 weeks after the chemokine pull was examined. The number of systemic memory gBT-I CD8⁺ T cells, although decreased compared to day 1 post pull (FIG. 1C) due to contraction of the T cell response, was similar regardless of immunization route or treatment (FIG. 3A). However, a significantly greater number and percentage of memory gBT-I CD8⁺ T cells was present in the genital tract of subcutaneously immunized mice treated with the chemokine (subcutaneously immunized plus pull) compared to chemokine untreated mice (subcutaneously immunized) (FIG. 3A, 3B). Despite significant recruitment during the effector phase (FIG. 1D), CD4⁺ T cells were not retained within the vagina long term (FIG. 3C), reminiscent of CD4⁺ T cell behavior after dermal HSV-1 infection in which the cells leave the site of infection to mediate immunosurveillance (Gebhardt et al., 2011, Nature 477:216-219). Thus, CD4⁺ T cells may require additional signals, such as those generated during HSV-2 infection (Zhu et al., 2009, Nature Med. 15:886-892; Iijima et al., 2008, J. Exp. Med. 205:3041-3052), to be retained long term within the vagina.

To investigate the stability of this tissue-resident population of memory gBT-I CD8⁺ T cells, T cell numbers at 12 weeks post-pull were also examined Donor gBT-I CD8⁺ T cell numbers in the vagina were significantly higher after prime and pull vaccination regimen than after subcutaneous immunization alone (FIG. 3D). Furthermore, the number of memory gBT-I CD8⁺ T cells did not decline between 4 weeks and 12 weeks (FIG. 3D), suggesting that this tissue-resident population was stable and retained long term. CD4⁺ T cell numbers in the vagina remained low at week 12 after prime and pull vaccination regimen, and were comparable to numbers detected at week 4 (FIG. 3E). Thus, a single chemokine pull given to mice during the effector phase is sufficient to establish a long-term population of tissue-resident memory CD8⁺ T cells, but not CD4⁺ T cells, within the vagina.

Tissue-resident memory T cells are effective in mediating immunity against local infections (Gebhardt et al., 2009, Nature Immunol. 10:524-530; Jiang et al., 2012, Nature 483:227-231). HSV-2 spreads from its initial replication site at the epithelium to the innervating neurons, and subsequently establishes latency within the dorsal root ganglia (DRG) (Koelle & Corey, 2008, Annu. Rev. Med. 59:381-395). Reactivation from latency leads to viral shedding and formation of genital lesions that are commonly associated with genital herpes (Koelle & Corey, 2008, Annu. Rev. Med. 59:381-395). Thus, preventing the spread of virus from the mucosal epithelium to the DRG is important in preventing disease and transmission of the virus.

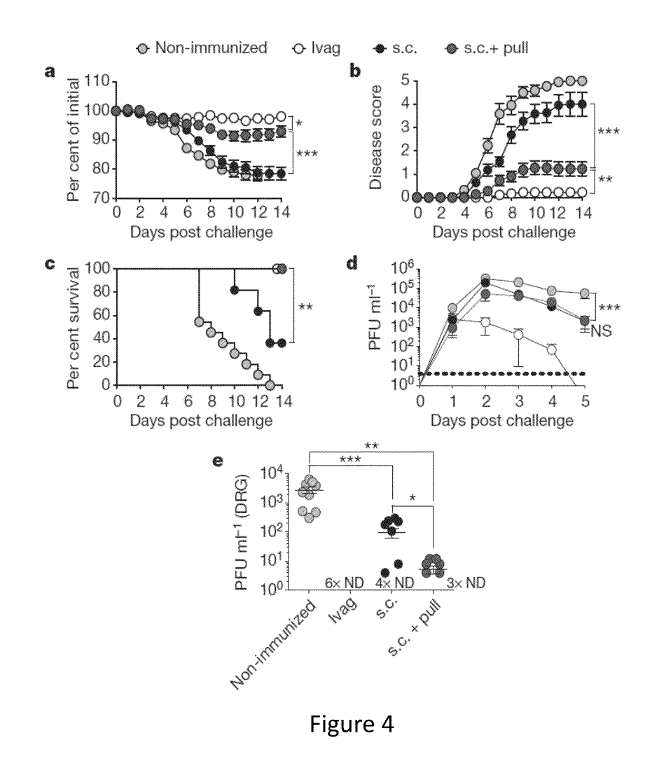

As a single chemokine pull administered after subcutaneous immunization is capable of establishing a population of tissue-resident memory CD8⁺ T cells within the vagina long term, whether the prime and pull vaccination regimen would provide enhanced immunity against genital HSV-2 infection was examined next. Mice were challenged intravaginally with a lethal dose of wild-type HSV-2 four weeks after the prime and pull vaccination regimen and monitored for disease and survival. Notably, mice treated with the chemokine pull (subcutaneously immunized plus pull) lost significantly less weight than either the non-immunized or subcutaneously immunized controls (FIG. 4A). Furthermore, prime and pull vaccination regimen almost completely prevented the development of clinical symptoms, which were observed in both non-immunized and subcutaneously immunized controls (FIG. 4B). Accordingly, mice treated with the chemokine pull had a 100% survival rate compared to the 36.3% survival rate of the subcutaneously immunized control (FIG. 4C).

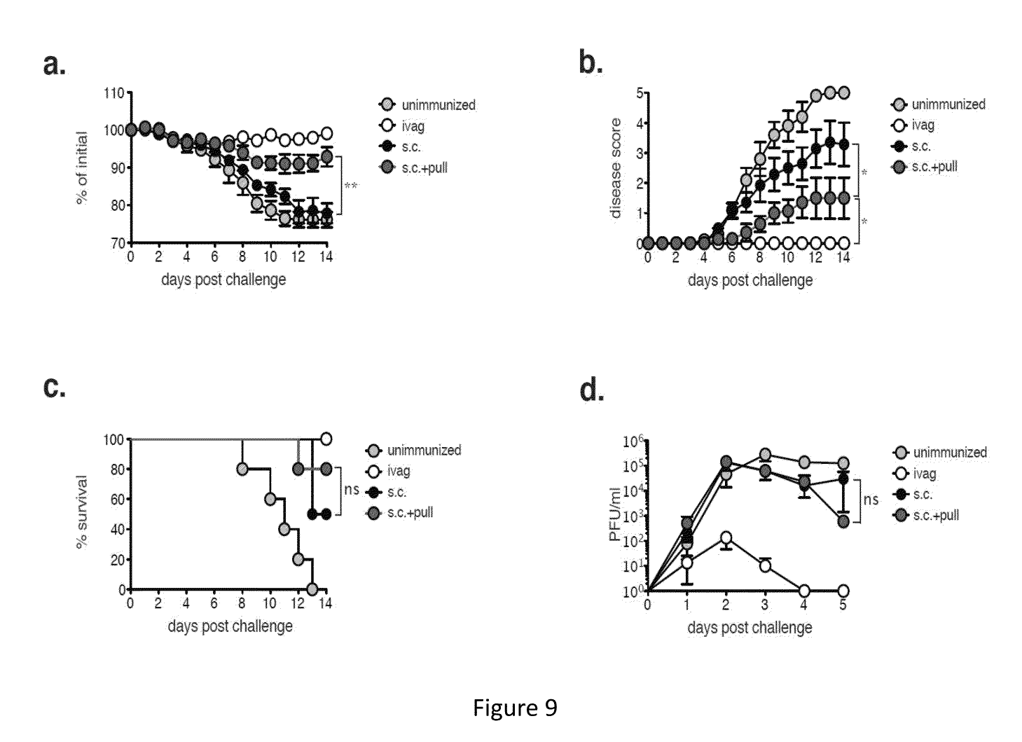

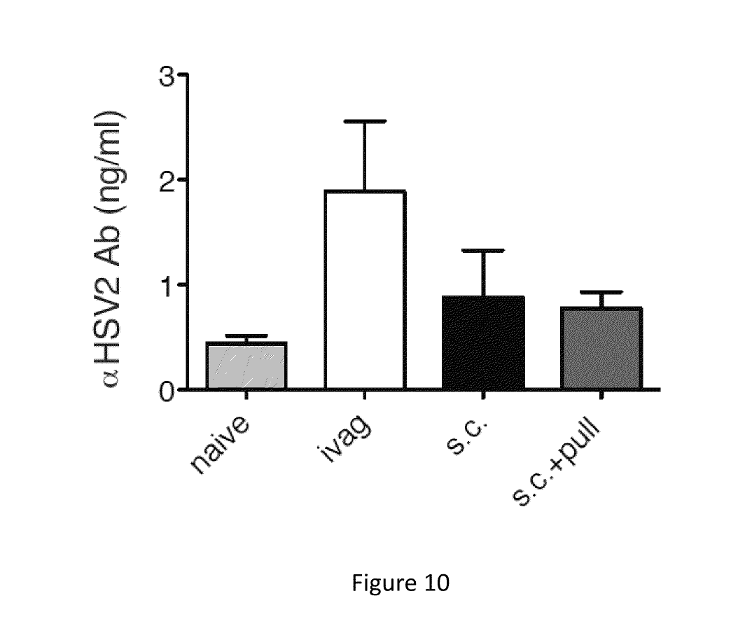

Upon challenge with wild-type HSV-2 four weeks post pull, mice immunized and chemokine-treated in the absence of T cell antigen receptor transgenic CD8⁺ T cells were also significantly protected from weight loss (FIG. 9A) and clinical disease (FIG. 9B), although a significant difference in survival rate was not observed (FIG. 9C). Anti-HSV antibody titres in the vagina were not significantly different between subcutaneously immunized controls and chemokine-treated mice (FIG. 10), suggesting that the control of viral challenge was probably T cell mediated. These results demonstrate that the addition of a chemokine pull to parenteral immunization could greatly enhance protective immunity against genital HSV-2 infection.

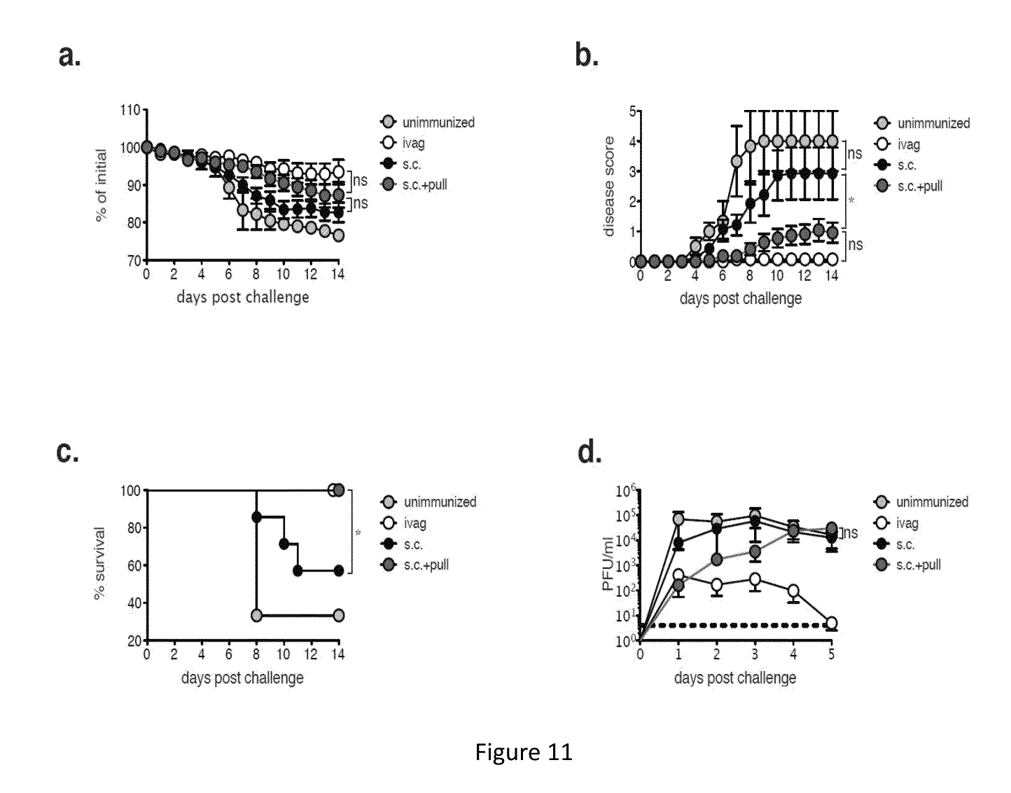

To test more stringently the long-term protection afforded by prime and pull vaccination regimen, mice 10-12 weeks post-pull were challenged. At this late time point, the prime and pull vaccination regimen group lost less weight compared to subcutaneously immunized controls (FIG. 11A), and were significantly protected from development of disease (FIG. 11B). Furthermore, at 2 weeks post challenge, the prime and pull vaccination regimen group had a survival rate of 100%, whereas subcutaneously immunized controls had a survival rate of 57% (FIG. 11C). Thus, the results show that the protection provided by prime and pull vaccination regimen lasts over time and remains robust up to 12 weeks after chemokine treatment.

To determine the mechanism by which the prime and pull vaccination regimen mediates protection from HSV-2 disease, viral replication within the genital mucosa was measured. Notably, no difference in virus titres from the vaginal secretion of subcutaneously immunized versus chemokine-treated (subcutaneously immunized plus pull) mice was found (FIGS. 4D, 9D, and 11D), indicating that protection was probably being mediated at a different location. As the more severe symptoms of clinical disease in mice are associated with viral replication in the peripheral nervous system (Parr & Parr, 2003, J. Neurovirol. 9:594-602), whether prime and pull vaccination regimen could protect the DRG against infection was examined next. When viral replication within the DRG was measured, it was found that mice treated with the chemokine pull had significantly lower virus titres than non-immunized mice (FIG. 4E). Furthermore, viral titres in the DRG of the prime and pull vaccination regimen group were significantly lower than that of subcutaneously immunized mice (FIG. 4E). Together, these data indicate that prime and pull vaccination regimen greatly reduces disease by controlling neuronal infection with HSV-2 rather than by controlling mucosal viral replication.

This study demonstrates that after conventional vaccination to generate a systemic T cell population (prime), a single topical treatment with chemokines applied vaginally (pull) can provide superior protection against genital herpes by, at least in part, decreasing the spread of virus from the mucosal epithelia into the neurons. Importantly, protection of neurons from HSV-2 infection by the prime and pull vaccination regimen may decrease reactivation and viral shedding, which may reduce disease and transmission. Although the exact role of T cells in controlling neuronal HSV-2 infection after prime and pull vaccination regimen is not yet clear, the local HSV-specific T cells may help to control entry of virus at the neuronal endings, or promote blockade of viral replication once inside the neurons. Furthermore, other studies have demonstrated that T cells recruited to the genital tract by inflammation alone can decrease viral replication at the mucosal surface (Mackay et al., 2012, Proc. Natl Acad. Sci. USA 109:7037-7042), suggesting that control of infection at the site of entry may be possible by optimizing prime and pull vaccination regimen. Thus, in addition to preventing reactivation of latent HSV (Khanna et al., 2004, Trends Immunol. 25:230-234), virus-specific memory T cells may be mobilized to control neuronal viral infection during primary infection. Although topical application in the genital tract of Toll-like receptor ligands such as imiquimod have been shown to be effective as a therapeutic approach (Perkins et al., 2011, Sex. Transm. Infect. 87:292-295), they may not be ideal vaccine candidates as they seem to be effective for only a short time after application and function through the induction of pro-inflammatory cytokines (Gill et al., 2008, Am. J. Reprod. Immunol. 59:35-43). The prime and pull vaccination strategy described herein provides an alternative to direct immunization of the genital tract, and establishes robust, long-term immunity with minimal local inflammation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of inducing an immune response in a subject in need thereof, and recruiting the immune response to an anatomic location of the subject, comprising the steps of:
a. parenterally administering to the subject at least one immunogen, wherein the at least one immunogen induces an immune response; and
b. locally administering to the anatomic location of the subject at least one chemokine, wherein the chemokine recruits the immune response to the anatomic location.

2. The method of claim 1, wherein the parenteral administration of the at least one immunogen is at least one selected from the group consisting of subcutaneous administration, intravenous administration, intramuscular administration, and intradermal administration.

3. The method of claim 1, wherein the local administration of the at least one chemokine is at least one selected from the group consisting of topical administration, subcutaneous administration, intramuscular administration, intradermal administration, intracranial administration and intratumoral administration.

4. The method of claim 1, wherein the chemokine is at least one selected from the group consisting of CXCL9, CXCL10 and CCL5.

5. The method of claim 1, wherein the anatomic location is an immunologically restrictive tissue.

6. The method of claim 1, wherein the anatomic location is at least one selected from the group consisting of the genital mucosa, a tumor, the skin, the central nervous system, the peripheral nervous system, the testes, the placenta, the eye, the intestine, and the lung airways.

7. The method of claim 1, wherein the immunogen is derived from a cancer cell.

8. The method of claim 1, wherein the immunogen is a derived from a tumor.

9. The method of claim 1, wherein the immunogen is derived from a pathogen selected from the group consisting of a virus, a bacterium, a fungi and a protozoan.

10. The method of claim 1, wherein the immunogen is at least one component of at least one selected from the group consisting of a live pathogenic organism, a live attenuated pathogenic organism, an inactivated pathogenic organism, and a dead pathogenic organism.

11. The method of claim 1, wherein the immunogen is at least one selected from the group consisting of a peptide, a polypeptide, and a polynucleotide encoding a polypeptide.

12. The method of claim 11, wherein the polynucleotide encoding a polypeptide is at least one selected from RNA and DNA.

13. The method of claim 11, wherein the polynucleotide encoding a polypeptide is a DNA vaccine.

14. The method of claim 9, wherein the subject is not currently infected with the pathogen and the immune response is a protective immune response.

15. The method of claim 9, wherein the subject is currently infected with the pathogen and the immune response is a therapeutic immune response.

16. The method of claim 7, wherein the subject does not currently have cancer and the immune response is a protective immune response.

17. The method of claim 7, wherein the subject currently has cancer and the immune response is a protective immune response.

18. The method of claim 1, wherein the immune response comprises a humoral immune response.

19. The method of claim 1, wherein the immune response comprises at least one antibody.

20. The method of claim 1, wherein the immune response comprises at least one antibody that specifically binds to the immunogen.

21. The method of claim 1, wherein the immune response comprises a cell-mediated immune response.

22. The method of claim 1, wherein the immune response comprises at least one activated immune cell.

23. The method of claim 1, wherein the activated immune cell is a CD4+ T cell.

24. The method of claim 1, wherein the activated immune cell is a CD8+ T cell.

25. The method of claim 1, wherein the activated immune cell is a CXCR3+ T cell.

26. The method of claim 1, wherein the activated immune cell is a CXCR3+CD4+ T cell.

27. The method of claim 1, wherein the activated immune cell is a CXCR3+CD8+ T cell.

28. The method of claim 1, wherein the activated immune cell is a CCR5+ T cell.

29. The method of claim 1, wherein the activated immune cell is a CCR5+CD4+ T cell.

30. The method of claim 1, wherein the activated immune cell is a CCR5+CD8+ T cell.

31. The method of claim 1, wherein the subject is human.


SEQUENCE LISTING (2 sequences)

SEQ ID NO: 1 - DNA, 18 bp, Artificial Sequence
  synthesized primer
agaccagggccgctgatc

SEQ ID NO: 2 - DNA, 19 bp, Artificial Sequence
  synthesized primer
gcgctggacctccgtgtag